(19) United States
(12) Patent Application Publication
(10) Pub. No.: US 2026/0115027 A1
(43) Pub. Date: Apr. 30, 2026

(54) BRAIDED DISTAL OUTER SHEATH FOR STENT DELIVERY SYSTEM

(71) Applicant: BOSTON SCIENTIFIC SCIMED, INC., Maple Grove, MN (US)

(72) Inventors: Anthony Frank Tassoni, JR., Andover, MN (US); Enda Connaughton, Galway (IE); Yvonne Brennan, Galway (IE)

(73) Assignee: BOSTON SCIENTIFIC SCIMED, INC., Maple Grove, MN (US)

(21) Appl. No.: 19/374,532

(22) Filed: Oct. 30, 2025

Related U.S. Application Data
(60) Provisional application No. 63/714,679, filed on Oct. 31, 2024.

(51) Int. Cl. A61F 2/966; A61F 2/962

(52) U.S. Cl. CPC A61F 2/966; A61F 2002/9623

(57) ABSTRACT

A stent delivery system includes an inner member with a distal end region including a visual marker, a stent surrounding the distal end region of the inner member including the visual marker, and an outer sheath slidingly surrounding the stent and the inner member. The outer sheath includes a braided reinforcement layer extending from a distal end to a proximal end of the outer sheath, and the outer sheath includes a proximal region in which the braided reinforcement layer has a first braid density, a distal region in which the braid reinforcement layer has a second braid density lower than the first braid density, and a transition region between the proximal region and the distal region. The distal region of the outer sheath is visually translucent or transparent and overlays the stent, and the visual marker and stent are viewable through the distal region of the outer sheath.

### CROSS-REFERENCE TO RELATED APPLICATIONS

The application claims the benefit of U.S. Provisional Patent Application Serial No. 63/714,679, filed on October 31, 2024, the disclosure of which is incorporated herein by reference.

### TECHNICAL FIELD

The disclosure pertains to medical devices for delivering stents, and more particularly to endoscopic stent delivery systems, and methods for using such systems.

### BACKGROUND

A wide variety of medical devices have been developed for medical use including, for example, endoprostheses or stents such as biliary, gastric, enteral, and esophageal stents. These stents are often delivered endoscopically to a predetermined treatment site within a body cavity or lumen. There is a need to accurately position the stent at the treatment site to facilitate proper placement of the stent upon deployment from the stent delivery device. Thus, there is a need, just before deployment and during deployment, to ascertain the location of the stent relative to the intended placement site. Of the known medical devices for delivering stents and associated methods, each has certain advantages and disadvantages. There is an ongoing need to provide alternative stent delivery devices and systems, as well as alternative methods for manufacturing and using the same.

### SUMMARY

This disclosure provides design, material, manufacturing method, and use alternatives for stent delivery devices. An example stent delivery system includes an inner member having a distal end region including a visual marker, a self-expanding stent disposed around the distal end region of the inner member and over the visual marker, an outer sheath slidingly disposed over the self-expanding stent and the inner member, the outer sheath including a braided reinforcement layer extending from a distal end of the outer sheath to a proximal end of the outer sheath. The outer sheath includes a proximal region in which the braided reinforcement

layer has a first braid density, a distal region in which the braided reinforcement layer has a second braid density lower than the first braid density, a transition region between the proximal region and the distal region in which density of the braided reinforcement layer varies from the first braid density to the second braid density along a length of the transition region, wherein the distal region of the outer sheath is visually translucent or transparent and overlays the self-expanding stent, and wherein the visual marker on the inner member is viewable through the distal region of the outer sheath.

Alternatively or additionally to the embodiment above, the stent delivery system has a deployment force of 5 pound force (lbf) or less.

Alternatively or additionally to any of the embodiments above, the braided reinforcement layer is a single continuous tubular braid extending through the proximal region and the distal region.

Alternatively or additionally to any of the embodiments above, the visual marker on the inner member is a pigment distributed along at least the distal end region.

Alternatively or additionally to any of the embodiments above, the pigment is distributed evenly along an entirety of the distal end region of the inner member.

Alternatively or additionally to any of the embodiments above, the first braid density is between 45 and 150 pics per inch and the second braid density is between 5 and 25 pics per inch.

Alternatively or additionally to any of the embodiments above, the first braid density is about 52 pics per inch and the second braid density is about 12.5 pics per inch.

Alternatively or additionally to any of the embodiments above, the braided reinforcement layer comprises metal wires selected from the group consisting of stainless steel, nitinol, nickel alloys, titanium alloys, and cobalt-chromium alloys.

Alternatively or additionally to any of the embodiments above, the outer sheath further comprises a lubricious inner liner extending from the proximal end of the outer sheath to the distal end of the outer sheath.

Alternatively or additionally to any of the embodiments above, the lubricious inner liner comprises a fluoropolymer.

Alternatively or additionally to any of the embodiments above, the density of the braided reinforcement layer in the transition region varies continuously from the first braid density to the second braid density.

Alternatively or additionally to any of the embodiments above, the density of the braided reinforcement layer in the transition region changes abruptly from the first braid density to the second braid density.

Alternatively or additionally to any of the embodiments above, the proximal region of the outer sheath is opaque.

Alternatively or additionally to any of the embodiments above, at least a distal portion of the proximal region of the outer sheath includes a pigment.

Alternatively or additionally to any of the embodiments above, the visual marker on the inner member is a pigment distributed along at least the distal end region, and wherein the pigment in the distal end region of the inner member and the pigment in the proximal region of the outer sheath are different and result in different colors.

Another example stent delivery system includes an outer sheath having a proximal end and a distal end, an inner member disposed within the outer sheath, the inner member including at least one colored visual marker, a self-expanding stent disposed between the inner member and the outer sheath, the stent underlying a transparent distal region of the outer sheath. The outer sheath includes a proximal region including a proximal segment of a braided reinforcement layer with a first braid density, a visually transparent distal region including a distal segment of the braided reinforcement layer with a second braid density, wherein the second braid density is lower than the first braid density, wherein the distal segment of the braided reinforcement layer extends throughout the visually transparent distal region of the outer sheath, and wherein the at least one colored visual marker of the inner member is visible through the visually transparent distal region of the outer sheath including the distal segment of the braided reinforcement layer.

Alternatively or additionally to the embodiment above, the stent delivery system has a deployment force of 5 pound force (lbf) or less.

Alternatively or additionally to any of the embodiments above, the proximal segment of the braided reinforcement layer and the distal segment of the braided reinforcement layer are formed by is a single continuous braid extending through the proximal region and the distal region.

Alternatively or additionally to any of the embodiments above, the first braid density is between 45 and 150 pics per inch and the second braid density is between 5 and 25 pics per inch.

Another example stent delivery system includes an outer sheath having a proximal region, a distal region, and a lumen extending therebetween, the outer sheath including a braided reinforcement layer extending through both the proximal region and the distal region, an inner member slidably disposed within the lumen of the outer sheath, a self-expanding stent surrounding a distal end region of the inner member and radially constrained within the distal region of the outer sheath, wherein the braided reinforcement layer has a first braid density throughout the proximal region, wherein the braided reinforcement layer has a second braid density throughout the distal region, wherein the second braid density is lower than the first braid density, wherein the braided reinforcement layer has a transition region between the first braid density and the second braid density in which the transition region of the braided reinforcement layer has a braid density that continuously varies from the first braid density to the second braid density throughout the transition region, wherein an entirety of the distal region of the outer sheath is visually transparent or translucent and overlays an entirety of the self-expanding stent, wherein the proximal region of the outer sheath is opaque, and wherein the inner member includes at least one colored visual marker that is visible through the distal region of the outer sheath including the braided reinforcement layer.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIGS. 1A and 1B are cross-sectional views of a prior art stent delivery system before and during stent deployment, respectively; and

FIGS. 2A and 2B are cross-sectional views of an example stent delivery system before and during stent deployment, respectively.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content transparently dictates otherwise. As used in this specification and the appended claims, the term “or” is generally employed in its sense including “and/or ” unless the content transparently dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal ”, “distal ”, “advance”, “withdraw”, variants thereof, and the like, may be generally considered with respect to the positioning, direction, and/or operation of various elements relative to a user/operator/manipulator of the device, wherein “proximal ” and “withdraw” indicate or refer to closer to or toward the user and “distal ” and “advance” indicate or refer to farther from or away from the user. In some instances, the terms “ proximal ”and “ distal ” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent ” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently – such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless transparently stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “ second ” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure.

Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified .

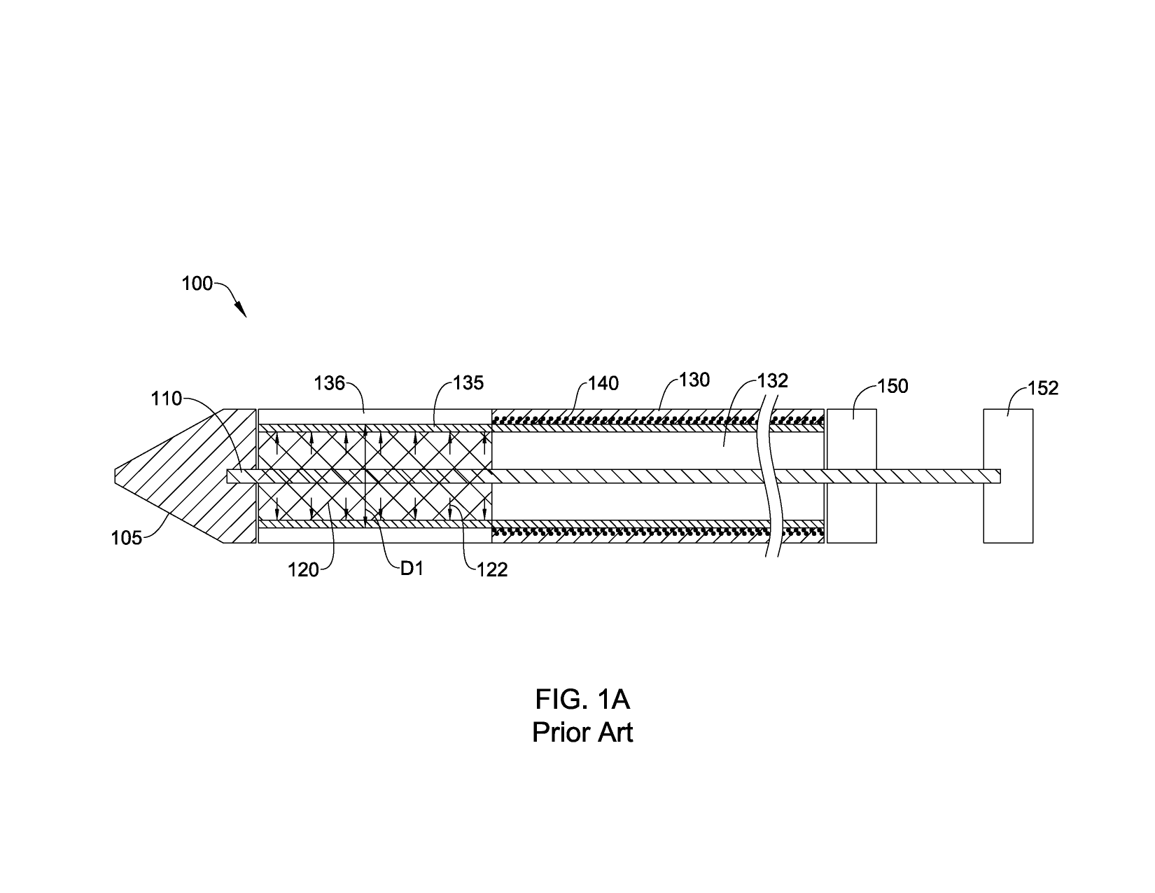

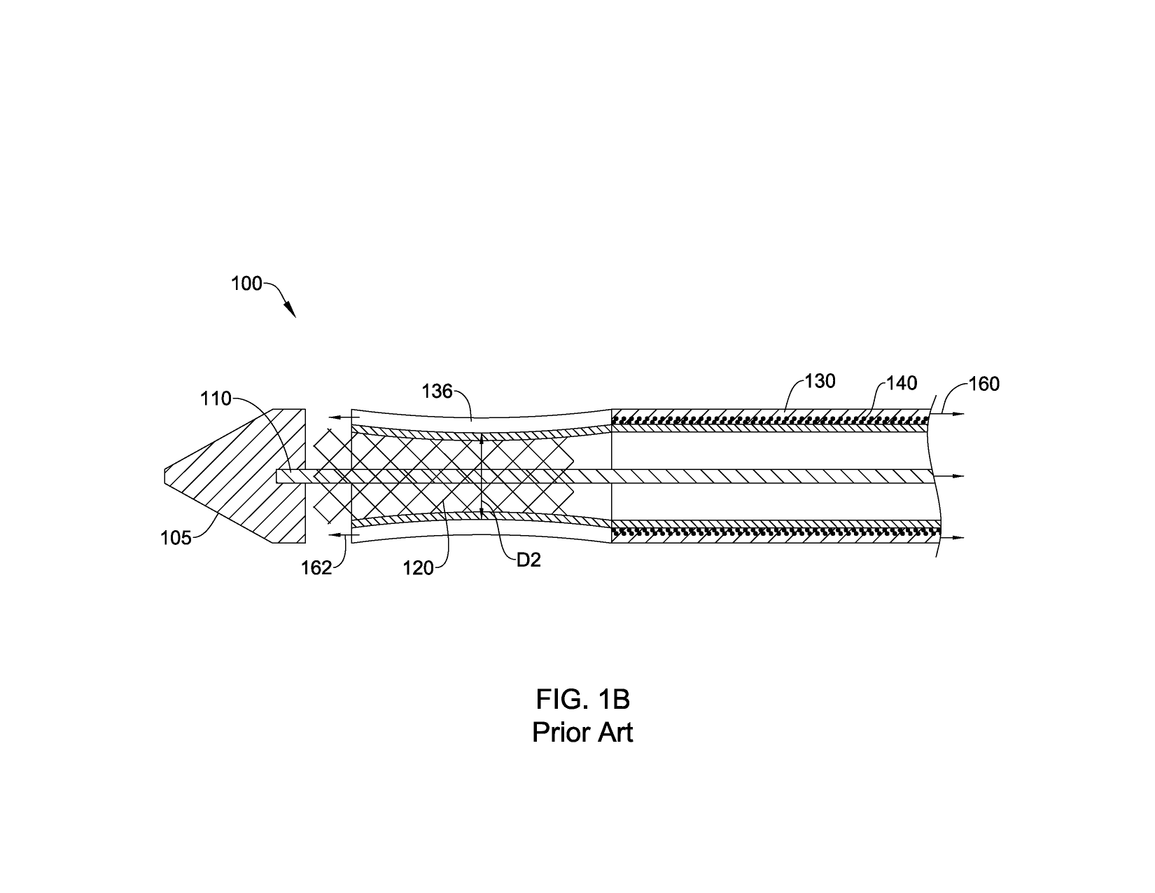

Endoscopic stent delivery systems 100 generally contain an inner member 110 with a distal tip 105 fixed to the distal end thereof, a stent 120, an outer sheath 130, a first handle 150, and a second handle 152, as shown in FIG. 1A. The stent 120 may be self-expanding and constrained within the lumen 132 of the outer sheath 130, and positioned between the outer sheath 130 and the inner member 110 for delivery to a treatment site. In this constrained configuration the stent 120 imposes a radial force, indicated by arrows 122, outward against the inner surface of the outer sheath 130. One known stent delivery system 100 includes an inner member 110 which is colored, while the outer sheath 130 includes a transparent distal region 136 in order to allow maximum visibility of the stent 120 and colored inner member 110 within the transparent distal region 136 during stent deployment. Endoscopy frequently uses visual markers instead of radiopaque markers, eliminating the need for fluoroscopy during stent delivery. The conventional outer sheath 130 often includes a braid 140 embedded therein, but the braid 140 does not extend over the stent 120 and through the transparent distal region 136 in order to avoid blocking visibility of the stent 120 and colored inner member 110 therewithin. The outer sheath 130 has an inner diameter D1 in the transparent distal region 136 that is substantially the same as in the proximal region with the braid 140. In some embodiments, the outer sheath 130 may include a lubricious liner 135 formed of a lubricous material, such as a polytetrafluoroethylene (PTFE).

To deploy the stent 120 in a desired location, the outer sheath 130 is pulled proximally relative to the stent 120 to uncover the stent 120 using the first, or distal handle 150 (affixed to a proximal end of the outer sheath 130) to release the stent 120, while the second, or proximal handle 152 (affixed to a proximal end of the inner member 110) is used to hold the inner member 110 in a constant position. The deployment force is highest at the beginning of the deployment as the outer sheath 130 begins to be moved proximally off the stent 120. The high deployment force must overcome friction between the outer sheath 130 and the stent 120, often caused by the knuckles or wire crossings of the stent 120 digging into the inner surface of the outer sheath 130 and holding the outer sheath 130 in place over the stent 120 (i.e., resisting proximal retraction of the outer sheath 130) until the deployment force overcomes this friction, at which point the outer sheath 130 may rapidly move proximally, including off the entire stent 120. The high deployment force may inadvertently cause the stent 120 to move distally as it is deployed, resulting in positioning errors. In general, the user desires to control the stent deployment, slowly and controllably withdrawing the outer sheath 130 in order to determine the stent 120 is in the desired position before completely uncovering the stent 120. Controlling the deployment also allows for easier repositioning and reconstrainment of the stent 120 when the outer sheath 130 has not been removed completely from the stent 120 and repositioning is desired.

There is also a longitudinal stress on the stent delivery system 100 which tends to elongate the outer sheath 130 (e.g., axially stretch the transparent distal region 136) and reduce the inner diameter D2 of the transparent distal region 136 of the outer sheath 130 over the stent 120, as shown in FIG. 1B. When the inner diameter of the distal region 136 of the outer sheath 130 is reduced during the initial stage of stent deployment, the frictional forces between the stent 120 and the inner surface of the outer sheath 130 increase, resulting in a need for an even greater proximal deployment force to be applied to the outer sheath 130, indicated by arrows 160, to initiate proximal movement of the outer sheath 130 relative to the stent 120. The proximal deployment force creates an equal and opposite force on the stent 120, indicated by arrows 162, which significantly increases the initial deployment force required to start moving the outer sheath 130 proximally off the stent 120, and which may lead to inadvertent movement of the stent 120 during the deployment process.

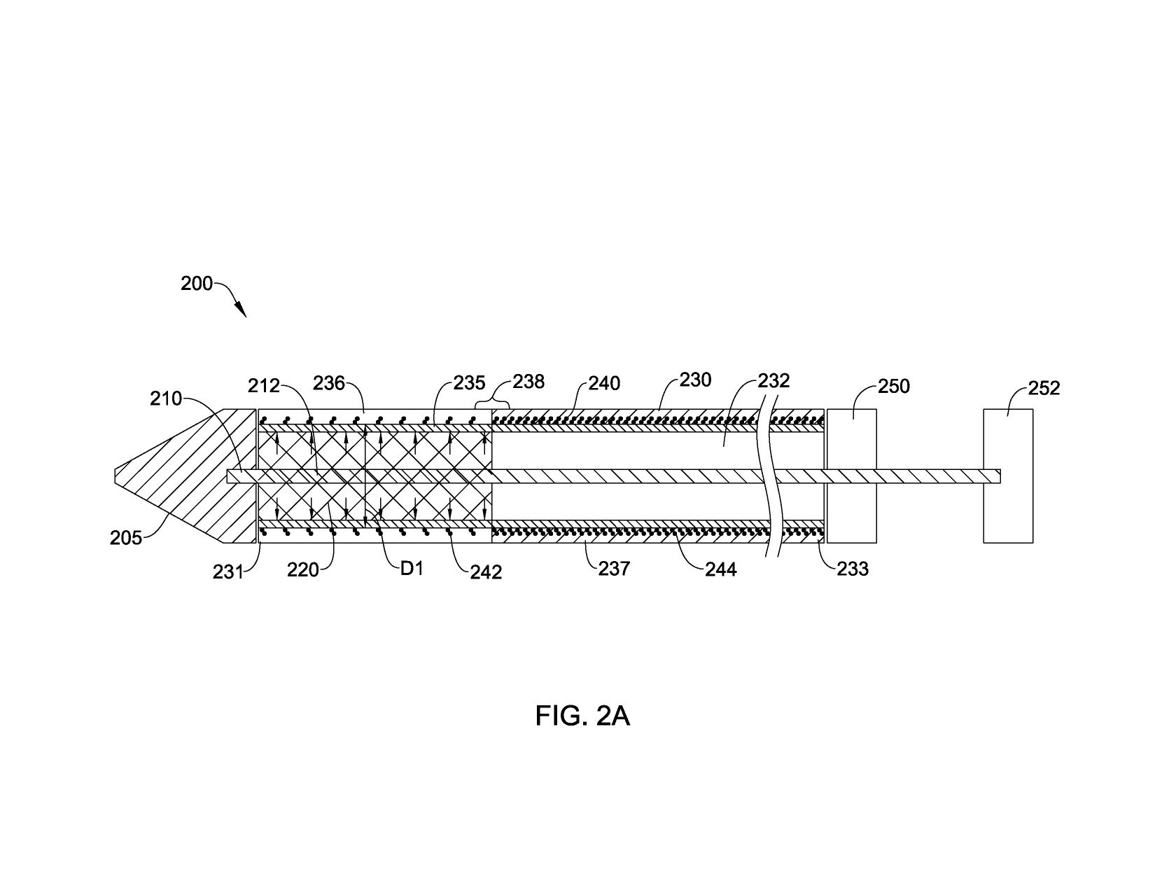

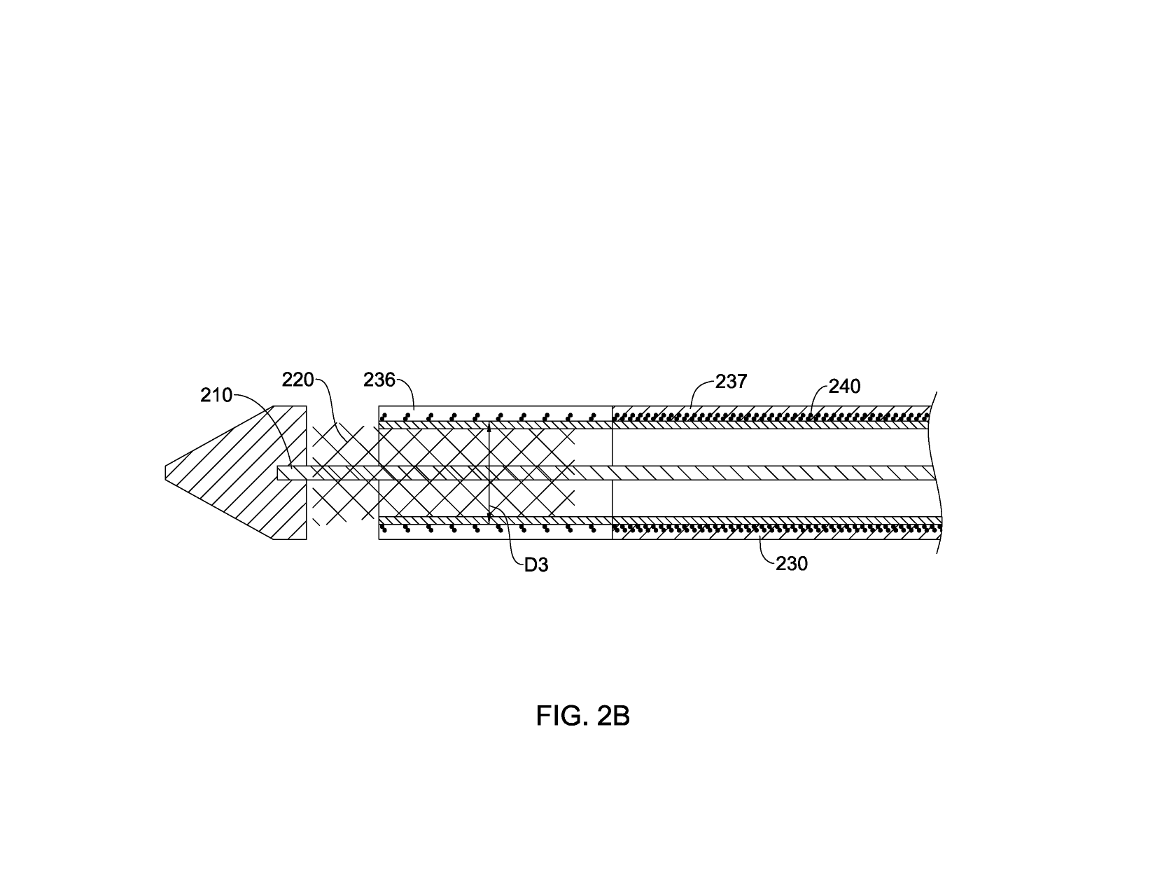

Once the outer sheath 130 begins to move proximally, the needed deployment force decreases quickly, which may result in the stent 120 being uncovered and deployed faster than desired, hindering precise positioning and/or repositioning of the stent 120. Users often experience high stress anticipating when the outer sheath 130 will start to move during the deployment process. Lower deployment force would equate to lower user stress during the procedure, more precise positioning of the stent 120, and easier reconstrainment and repositioning when needed. The stent delivery system 200 described below and depicted in FIGS. 2A and 2B achieves a lower deployment force while maintaining visualization of the stent and any colored inner member.

The stent delivery system 200 includes an elongate inner member 210, a stent 220 surrounding a distal end region 212 of the inner member 210, a distal tip 205 formed or secured at the distal end of the inner member 210, and an outer sheath 230 slidingly disposed over the stent 220 and the inner member 210. The stent delivery system 200 may also include a first, distal handle 250 secured to a proximal end of the outer sheath 230 and a second, proximal handle 252 secured to the proximal end of the inner member 210, as shown in FIG. 2A. The second, proximal handle 252 may be used hold a position of the inner member 210 and the stent 220 during delivery while the first, distal handle 250 may be moved proximally toward the second handle 252 to proximally retract the outer sheath 230 from the stent 220. The outer sheath 230 may be an elongate tube defining a lumen 232 through which the inner member 210 axially slides. The stent 220 may be radially self-expanding, surrounding the distal end region 212 of the inner member 210 and radially constrained by the outer sheath 230 in a radially reduced, axially elongated state. The stent 220 may be radially self-expanding, in that once free of (i.e., unconstrained by) the outer sheath 230, the stent 220 may expand radially, while in some cases shortening axially, to a deployed configuration in which the stent diameter is greater than the diameter of the outer sheath 230. The stent 220 may be formed of one or more, or a plurality of wires or filaments which is/are wound, interwoven, braided, knitted, or otherwise formed to define a tubular scaffold.

In some embodiments, the distal end region 212 of the inner member 210 may include a visual marker. The visual marker may be achieved by coloring the entire distal end region 212 underlying the stent 220, for example by the addition of a pigment incorporated into the polymer material forming the inner member 210 during manufacturing. The pigment may be distributed evenly along at least the distal end region 212. In other instances, the visual marker may be provided by the addition of a paint/ink layer applied to the inner member 210, for example. The stent 220 is thus disposed over (i.e., surrounds) the visual marker. Alternatively, colored, visible markings may be added to the distal end region 212 of the inner member 210 after manufacturing.

The outer sheath 230 may include a translucent or transparent distal region 236 overlying and surrounding the stent 220. The transparent distal region 236 may be made of a material exhibiting a high transmissivity of energy in the visible spectrum. In some embodiments, the distal region 236 may be translucent, in the sense that at least 25% of the energy in the visible spectrum impinging directly upon the outer sheath 230 in this area is transmitted through the distal region 236 to the interior of the outer sheath 230. The visual marker of the inner member 210 may be viewable through the transparent distal region 236 of the outer sheath 230.

The proximal region 237 of the outer sheath 230 may be opaque. In some embodiments, the proximal region 237 may be colored, such as with a pigment incorporated in the polymer forming the proximal region 237. The pigment in the distal end region 212 of the inner member 210 and the pigment in the proximal region 237 of the outer sheath 230 may be different and result in different colors. In one example, the pigment in the distal end region 212 of the inner member 210 results in the distal end region 212 of the inner member 210 being yellow and the pigment in the proximal region 237 of the outer sheath 230 results in the proximal region 237 being blue. Other combinations of colors are also contemplated.

The transition between the transparent distal region 236 and the opaque proximal region 237 may be abrupt, with a distinct junction between the transparent distal region 236 and the opaque proximal region 237. This distinct junction may aid the user in positioning the stent 220 in a desired location. The outer sheath 230 may include a lubricious inner liner 235 including a fluoropolymer, such as PTFE, or other lubricious material bonded to the inner surface of the outer sheath 230. The inner liner 235 may extend throughout the distal region 236 and proximally across the junction and into the proximal region 237. In some instances, the inner liner 235 may extend the entire length of the outer sheath 230, from a distal end 231 of the outer sheath 230 to a proximal end 233 of the outer sheath 230, and may be substantially translucent to transparent. In some instances, the inside surface of inner liner 235 may be coated with silicone or other lubricious material, to provide a low-friction surface to contact the stent 220.

The outer sheath 230 may include a braided reinforcement layer 240 embedded therein. The braided reinforcement layer 240 may be completely embedded such that no portion of the braided reinforcement layer 240 extends radially beyond the inner and outer surfaces of the outer sheath 230. The outer sheath 230, including the transparent distal region 236, the opaque proximal region 237, the inner liner 235, and the braided reinforcement layer 240 may be bonded together to form an integral tubular structure. The braided reinforcement layer 240 may be a tubular braid formed of helically wound intersecting filaments of stainless steel, nitinol, a nickel-based alloy, a titanium-based alloy, a cobalt-based alloy, or other suitable metal. In other instances, the braided reinforcement layer 240 may be formed of helically wound intersecting polymeric filaments made of, for example, polyimide, Kevlar® , Vectran® , etc. The braided reinforcement layer 240 may extend throughout the distal region 236 and proximally across the junction and into the proximal region 237. In some instances, the braided reinforcement layer 240 may extend continuously the entire length of the outer sheath 230, from the distal end 231 to the proximal end 233. In other words, the filaments forming the braided reinforcement layer 240 may each extend continuously along the entire length of the outer sheath 230, from the distal end 231 to the proximal end 233. In some embodiments, the braided reinforcement layer 240 may be a tubular braid formed from a plurality of filaments, such as stainless-steel wires, each having a diameter of about 0.015 inches. In one arrangement, 32 filaments or wires may be wound helically, interbraided in a one-over-one-under or two-over-two-under pattern. The braided reinforcement layer 240 provides a reinforcing structure that increases the columnar strength of the outer sheath 230, and also increases radial stability and resistance to kinking when the outer sheath 230 is bent.

The braided reinforcement layer 240 may have a first braid pattern having a first braid density in the proximal region 237 of the outer sheath 230, a second braid pattern having a second braid density in the distal region 236, and a transition region 238 between the proximal region 237 and the distal region 236. The second braid density may be lower than the first braid density. The braid density may vary from the first braid density to the second braid density along the length of the transition region 238. In some embodiments, the braid density may vary continuously from the first braid density to the second braid density. The braided reinforcement layer 240 may be only a single continuous braid formed from a plurality of wires or ribbons that each extend continuously the entire length of the braided reinforcement layer 240 from the proximal end 233 of the outer sheath 230 to the distal end 231 of the outer sheath 230. The wires or filaments forming the braided reinforcement layer 240 may have any cross-sectional shape, including, but not limited to, round, flat, oval and D-shaped. Braid density may be measured in pic-per-inch (ppi). The pic-per-inch or ppi count of a braid pattern refers to the quantity of wire crossings per inch. In general, higher ppi braids or braid patterns are more flexible while lower ppi braids or braid patterns have increased longitudinal stiffness.

In the embodiment shown in FIGS. 2A and 2B, the first braid density in the proximal region 237 of the braid 240 may be between 45 ppi and 150 ppi, and the second braid density in the distal region 236 may be between 5 ppi and 25 ppi. In one example, the first braid density throughout the proximal region 237 is in the range of about 45 -55 ppi, or about 52 ppi, and the second braid density throughout the distal region 236 is in the range of about 10 – 15 ppi, or about 12.5 ppi. The braided reinforcement layer 240 may thus include a low ppi section 242 throughout the distal region 236 (e.g., 10 -15 ppi) and a high ppi section 244 throughout the proximal region 237 (e.g., 45-55 ppi). The low ppi section 242 may extend the entire length of the transparent distal region 236, and the high ppi section 244 may extend proximally from the low ppi section 242 to the proximal end 233 of the outer sheath 230, for example. The braid density in the proximal region 237, and thus the high ppi section 244, may be continuous throughout the entire length of the proximal region 237, and the braid density in the distal region 236, and thus the low ppi section 242, may be continuous throughout the entire length of the distal region 236, with the transition region 238 being the only portion of the outer sheath 230 in which the braid density is variable. The length of the low ppi section 242 may be sized to cover the entire length of the stent 220 in its radially constrained configuration within the outer sheath 230, and in some instances may be between 100 millimeters (mm) and 300 mm, for example. The length of the transition region 238 between the low ppi section 242 and the high ppi section 244 may be between 0.5 inch and 1.5 inches. The transition in ppi may be abrupt or gradual and continuous. The transition region 238 may extend completely within the transparent distal region 236, may extend completely within the opaque proximal region 237, or it may straddle the junction and extend into both the transparent distal region 236 and the opaque proximal region 237, as shown in FIG. 2A, with the midpoint of the transition between the low ppi section 242 and the high ppi section 244 positioned at the junction between transparent and opaque regions of the outer sheath 230. The low ppi section 242 of the braided reinforcement layer 240 allows for the stent 220 and inner member 210, which may be colored, to be visualized through the braided reinforcement layer 240 during stent delivery and deployment. The low ppi section 242 of the braided reinforcement layer 240 may have spaced-apart wires or filaments, allowing the user to see through the braided reinforcement layer 240.

The transparent distal region 236 may have a length that is equal to or greater than the length of the stent 220 compressed for deployment and loaded into the distal region 236 of the outer sheath 230 of the stent delivery system 200. Further, the outer sheath 230 with the braided reinforcement layer 240 in the distal region 236 reduces the longitudinal stress on the stent delivery system 200 so the distal region 236 is not stretched or elongated and its inner diameter D3 is not reduced during the initial stage of stent deployment, as shown in FIG. 2B. Accordingly, the inner diameter D3 of the transparent distal region 236 with the low ppi section 242 of the braided reinforcement layer 240 in the deployment configuration in which the outer sheath 230 moves proximally to expose the stent 220 (see FIG. 2B) is substantially the same as the inner diameter D1 of the transparent distal region 236 with the low ppi section 242 of the braided reinforcement layer 240 in the loaded stent delivery system 200 (see FIG. 2A).

The low ppi section 242 of the braided reinforcement layer 240 in the transparent distal region 236 of the outer sheath 230 results in approximately a 50% reduction in deployment force by lowering elongation and inner diameter reduction of the outer sheath 230, as compared to the conventional outer sheath 130 with no braid in the transparent distal region 136. For example, the stent delivery system 200 may have a deployment force of 5 pound force (lbf) or less, as compared to the stent delivery system 100 without a braid in the transparent distal region 136, which comparatively has a deployment force of between 8-10 lbf. The deployment force refers to the amount of proximally directed axial force applied to the first handle 250 necessary to effect proximal retraction of the outer sheath 230 relative the stent 220 to uncover the stent 220. This lower deployment force equates to a much better user experience during a medical procedure and reduces inadvertent movement of the stent and inner member 210 during the deployment of the stent 220.

The outer sheath 230 may be manufactured according to traditional methods, which generally involve assembling each outer sheath separately by hand. For example, the inner liner 235 may be formed or otherwise disposed on a mandrel. A braided reinforcement layer 240 may be formed on the inner liner 235 or formed separately and thereafter loaded onto the inner liner 235. The opaque and transparent sections of the outer sheath 230 may be extruded over the braided reinforcement layer 240, or formed separately as discrete tubular segments and then loaded over the braided reinforcement layer 240 and bonded thereto through a reflow process. Alternatively, the single continuous braid structure with variable braid density may allow for the outer sheath 230 to be manufactured using a reel-to-reel assembly process. For example, a 1,000 foot spool of liner 235 tubing may have the braid 240 formed over it, followed by extruding the polymer layers forming the opaque 237 and transparent 236 sections of the outer sheath 230. Individual catheter shafts lengths may be cut from the spooled construct. Reel-to-reel manufacturing may provide a significantly faster and cheaper assembly of the outer sheath 230.

It will be understood that the dimensions described in association with the above figures are illustrative only, and that other dimensions are contemplated. The materials that can be used for the various components of the stent delivery system 200 and the various elements thereof disclosed herein may include those commonly associated with medical devices.

In some embodiments, the stent delivery system 200 (and variations, systems or components thereof disclosed herein) may be made from a metal, metal alloy, ceramics, zirconia, polymer (some examples of which are disclosed below), a metal-polymer composite, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; cobalt chromium alloys, titanium and its alloys, alumina, metals with diamond-like coatings (DLC) or titanium nitride coatings, other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

In some embodiments, the stent delivery system 200 (and variations, systems or components thereof disclosed herein) and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene- b -isobutylene- b -styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

In at least some embodiments, portions or all of the stent delivery system 200 (and variations, systems or components thereof disclosed herein) may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the stent delivery system 200 (and variations, systems or components thereof disclosed herein). Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the stent delivery system 200 (and variations, systems or components thereof disclosed herein) to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A stent delivery system comprising:
an inner member having a distal end region including a visual marker;
a self-expanding stent disposed around the distal end region of the inner member and over the visual marker;
an outer sheath slidingly disposed over the self-expanding stent and the inner member, the outer sheath including a braided reinforcement layer extending from a distal end of the outer sheath to a proximal end of the outer sheath, the outer sheath comprising:
a proximal region in which the braided reinforcement layer has a first braid density;
a distal region in which the braided reinforcement layer has a second braid density lower than the first braid density;
a transition region between the proximal region and the distal region in which density of the braided reinforcement layer varies from the first braid density to the second braid density along a length of the transition region;
wherein the distal region of the outer sheath is visually translucent or transparent and overlays the self-expanding stent; and
wherein the visual marker on the inner member is viewable through the distal region of the outer sheath.

2. The stent delivery system of claim 1, wherein the stent delivery system has a deployment force of 5 pound force (lbf) or less.

3. The stent delivery system of claim 1, wherein the braided reinforcement layer is a single continuous tubular braid extending through the proximal region and the distal region.

4. The stent delivery system of claim 1, wherein the visual marker on the inner member is a pigment distributed along at least the distal end region.

5. The stent delivery system of claim 4, wherein the pigment is distributed evenly along an entirety of the distal end region of the inner member.

6. The stent delivery system of claim 1, wherein the first braid density is between 45 and 150. pics per inch and the second braid density is between 45 and 150. pics per inch.

7. The stent delivery system of claim 6, wherein the first braid density is about 52 pics per inch and the second braid density is about 12.5 pics per inch.

8. The stent delivery system of claim 1, wherein the braided reinforcement layer comprises metal wires selected from the group consisting of stainless steel, nitinol, nickel alloys, titanium alloys, and cobalt-chromium alloys.

9. The stent delivery system of claim 1, wherein the outer sheath further comprises a lubricious inner liner extending from the proximal end of the outer sheath to the distal end of the outer sheath.

10. The stent delivery system of claim 9, wherein the lubricious inner liner comprises a fluoropolymer.

11. The stent delivery system of claim 1, wherein the density of the braided reinforcement layer in the transition region varies continuously from the first braid density to the second braid density.

12. The stent delivery system of claim 1, wherein the density of the braided reinforcement layer in the transition region changes abruptly from the first braid density to the second braid density.

13. The stent delivery system of claim 1, wherein the proximal region of the outer sheath is opaque.

14. The stent delivery system of claim 13, wherein at least a distal portion of the proximal region of the outer sheath includes a pigment.

15. The stent delivery system of claim 14, wherein the visual marker on the inner member is a pigment distributed along at least the distal end region, and wherein the pigment in the distal end region of the inner member and the pigment in the proximal region of the outer sheath are different and result in different colors.

16. A stent delivery system comprising:
an outer sheath having a proximal end and a distal end;
an inner member disposed within the outer sheath, the inner member including at least one colored visual marker;
a self-expanding stent disposed between the inner member and the outer sheath, the stent underlying a transparent distal region of the outer sheath;
wherein the outer sheath comprises:
a proximal region including a proximal segment of a braided reinforcement layer with a first braid density;
a visually transparent distal region including a distal segment of the braided reinforcement layer with a second braid density, wherein the second braid density is lower than the first braid density;
wherein the distal segment of the braided reinforcement layer extends throughout the visually transparent distal region of the outer sheath; and
wherein the at least one colored visual marker of the inner member is visible through the visually transparent distal region of the outer sheath including the distal segment of the braided reinforcement layer.

17. The stent delivery system of claim 16, wherein the stent delivery system has a deployment force of 5 pound force (lbf) or less.

18. The stent delivery system of claim 16, wherein the proximal segment of the braided reinforcement layer and the distal segment of the braided reinforcement layer are formed by is a single continuous braid extending through the proximal region and the distal region.

19. The stent delivery system of claim 16, wherein the first braid density is between 45 and 150. pics per inch and the second braid density is between 45 and 150. pics per inch.

20. A stent delivery system comprising:
an outer sheath having a proximal region, a distal region, and a lumen extending therebetween, the outer sheath including a braided reinforcement layer extending through both the proximal region and the distal region;
an inner member slidably disposed within the lumen of the outer sheath;
a self-expanding stent surrounding a distal end region of the inner member and radially constrained within the distal region of the outer sheath;
wherein the braided reinforcement layer has a first braid density throughout the proximal region;
wherein the braided reinforcement layer has a second braid density throughout the distal region, wherein the second braid density is lower than the first braid density;
wherein the braided reinforcement layer has a transition region between the first braid density and the second braid density in which the transition region of the braided reinforcement layer has a braid density that continuously varies from the first braid density to the second braid density throughout the transition region;
wherein an entirety of the distal region of the outer sheath is visually transparent or translucent and overlays an entirety of the self-expanding stent;
wherein the proximal region of the outer sheath is opaque; and
wherein the inner member includes at least one colored visual marker that is visible through the distal region of the outer sheath including the braided reinforcement layer.
